(19) United States
(12) Patent Application Publication
(10) Pub. No.: US 2026/0367050 A1
(43) Pub. Date: Nov. 19, 2026

(54) USE OF HYDROXYAPATITE IN PRETREATMENT OF FRUIT JUICE, PRETREATMENT METHOD OF SAMPLE, AND DETECTION METHOD OF LACTONE COMPOUND IN FRUIT JUICE

(71) Applicant: Guangzhou Flower Flavours Fragrances Co., Ltd., Guangzhou (CN)

(72) Inventors: Guoyin LAI, Guangzhou (CN); Mengqian ZHANG, Guangzhou (CN); Dunming XU, Guangzhou (CN); Fuhong CHEN, Guangzhou (CN); Qingbiao ZHAO, Guangzhou (CN); Tengfei DING, Guangzhou (CN); Yong CHEN, Guangzhou (CN); Yingyao HE, Guangzhou (CN); Weiquan YANG, Guangzhou (CN); Jiayou YANG, Guangzhou (CN)

(73) Assignee: Guangzhou Flower Flavours Fragrances Co., Ltd., Guangzhou (CN)

(21) Appl. No.: 19/399,689

(22) Filed: Nov. 25, 2025

(30) Foreign Application Priority Data
Mar. 12, 2025 (CN) 202510296015.5

(51) Int. Cl. G01N 30/34; G01N 1/38; G01N 1/40; G01N 30/02; G01N 30/72; G01N 33/14

(52) U.S. Cl. CPC G01N 30/34; G01N 1/38; G01N 1/4077; G01N 30/7206; G01N 33/14; G01N 2001/4088; G01N 2030/025; G01N 2030/347

(57) ABSTRACT

Provided are use of hydroxyapatite in pretreatment of a fruit juice, a pretreatment method of a sample, and a detection method of a lactone compound in a fruit juice. A pretreatment method of a sample, includes: subjecting a fruit juice to be tested, acetonitrile, first magnesium sulfate, and sodium chloride to a first mixing, and then a first vortexing and a first centrifugation in sequence to obtain a first liquid phase; subjecting the first liquid phase, second magnesium sulfate, and hydroxyapatite to a second mixing, and then a second vortexing and a second centrifugation in sequence to obtain a second liquid phase; and filtering the second liquid phase through an organic filter membrane to obtain a test solution.

### CROSS REFERENCE TO RELATED APPLICATION

This patent application claims the benefit and priority of Chinese Patent Application No. 202510296015.5 filed with the China National Intellectual Property Administration on Mar. 12, 2025, the disclosure of which is incorporated by reference herein in its entirety as part of the present application.

### TECHNICAL FIELD

The present disclosure belongs to the technical field of chemical analysis, and in particular relates to use of hydroxyapatite in pretreatment of a fruit juice, a pretreatment method of a sample, and a detection method of a lactone compound in a fruit juice.

### BACKGROUND

In the realm of ready-to-drink beverages, consumers highly favor products containing natural ingredients of fruit juice, such as not-from-concentrate (NFC) fruit juice. However, the addition of low-cost, chemically synthesized fruit fragrances to beverages as a means of passing off as genuine is a recurring issue. Consequently, it is necessary to conduct authenticity verification tests on fruit juices. Currently, the commonly employed detection methods primarily include sensory evaluation, conventional physical and chemical methods (such as saccharide identification, organic acid identification, pectin substance determination, phenolic compound determination, and inorganic element identification), high-performance liquid chromatography, gas chromatography, mass spectrometry, etc. Among these, the accuracy of sensory evaluation and conventional physical and chemical methods is relatively poor. While high-performance liquid chromatography, gas chromatography, and mass spectrometry have significantly improved detection precision, their operations are notably complex. For instance, gas chromatography requires the use of fingerprint chromatograms to detect substances such as saccharides, organic acids, and aromatic components in fruit juice beverages. Additionally, during the detection process, it is necessary to prepare these substances into volatile derivatives for measurement.

### SUMMARY

In view of this, the present disclosure provides use of hydroxyapatite in pretreatment of a fruit juice, a pretreatment method of a sample, and a detection method of a lactone compound in a fruit juice. The pretreatment method in the present disclosure can efficiently separate a lactone compound in the to-be-tested species. By controlling the condition parameters, the separation of chiral lactone compound isomers can be achieved, improving detection efficiency while ensuring the accuracy of the detection results.

In order to solve the technical problems described above, the present disclosure provides use of hydroxyapatite as an adsorbent in pretreatment of a sample when detecting a lactone compound in a fruit juice using gas chromatography-mass spectrometry (GC-MS).

The present disclosure provides a pretreatment method of a sample, including the following steps:
subjecting a fruit juice to be tested, acetonitrile, first magnesium sulfate, and sodium chloride to a first mixing, and then a first vortexing and a first centrifugation in sequence to obtain a first liquid phase;
subjecting the first liquid phase, second magnesium sulfate, and hydroxyapatite to a second mixing, and then a second vortexing and a second centrifugation in sequence to obtain a second liquid phase; and
filtering the second liquid phase through an organic filter membrane to obtain a test solution.

In some embodiments, a mass ratio of the fruit juice to be tested to the first magnesium sulfate is in a range of 5:1.8 to 5:2.2; and
a mass ratio of the fruit juice to be tested to the sodium chloride is in a range of 5:1.8 to 5:2.2.

In some embodiments, the first vortexing is conducted for 0.8 min to 1.2 min; and
the first centrifugation is conducted at a rotation speed of 3,800 rpm to 4,200 rpm for 4 min to 6 min.

In some embodiments, a ratio of a mass of the second magnesium sulfate to a volume of the first liquid phase is in a range of 1 g: 8 mL to 1.4 g: 8 mL; and
a ratio of a mass of the hydroxyapatite to the volume of the first liquid phase is in a range of 380 mg: 8 mL to 420 mg: 8 mL.

In some embodiments, the second mixing is conducted under conditions for the second vortexing, and the second vortexing is conducted for 25 s to 35 s; and
the second centrifugation is conducted at a rotation speed of 3,800 rpm to 4,200 rpm for 4 min to 6 min.

In some embodiments, the organic filter membrane has an average pore size of 0.2 μm to 0.24 μm.

The present disclosure further provides a detection method of a lactone compound in a fruit juice, including the following steps:
subjecting a test solution to GC-MS detection to obtain a total ion chromatogram; and
obtaining a ratio of R configuration to S configuration of the lactone compound in the test solution based on the total ion chromatogram; where
the test solution is the test solution obtained by the pretreatment method as described above;
the lactone compound includes at least one selected from the group consisting of γ-valerolactone, γ-caprolactone, γ-heptalactone, γ-octalactone, γ-nonalactone, δ-nonalactone, γ-decalactone, δ-decalactone, γ-undecanolactone, δ-undecanolactone, γ-dodecalactone, and δ-dodecalactone; and
conditions for a gas chromatography detection in the GC-MS detection include: chromatographic column: CP-Chirasil Dex CB; split ratio: 10:1; and temperature rise program: initial column temperature: 40 °C, increase to 80 °C at a heating rate of 10 °C/min, then to 180 °C at a heating rate of 2 °C/min, and then to 200 °C at a heating rate of 10 °C/min, and hold at the 200 °C for less than 20 min.

In some embodiments, the conditions for the gas chromatography detection further include: injection volume: 1 μL; constant flow mode, with helium as a carrier gas at a flow rate of 0.8 mL/min to 1.2 mL/min; inlet temperature: 220 °C to 250 °C; and transfer line temperature: 200 °C.

In some embodiments, conditions for a mass spectrometry detection in the GC-MS detection include: using electron impact ionization, with an ionization energy of 70 eV; a temperature of an ion source and quadrupole rods being 230 °C and 150 °C, respectively; solvent delay: 5 min to 10 min; full scan mode with a mass scan range of 40 m/z to 400 m/z; and parameters for single ion monitoring mode being shown in Table 1:

TABLE 1
Parameters for single ion monitoring mode

| | Monitored | Start time | Quantitative | Qualitative | Qualitative |
|---|---|---|---|---|---|
| No. | compound | (min) | ion (m/z) | ion 1 (m/z) | ion 2 (m/z) |
| 1 | γ-Valerolactone | 10.0 | 56 | 41 | 85 |
| 2 | γ-Caprolactone | 15.3 | 85 | 57 | 70 |
| 3 | γ-Heptalactone | 20.8 | 85 | 56 | 100 |
| 4 | γ-Octalactone | 26.0 | 85 | 56 | 100 |
| 5 | γ-Nonalactone | 29.2 | 99 | 71 | 114 |
| 6 | δ-Nonalactone | 31.6 | 85 | 100 | 114 |
| 7 | γ-Decalactone | 34.0 | 99 | 71 | 114 |
| 8 | δ-Decalactone | 36.4 | 85 | 55 | 128 |
| 9 | γ-Undecanolactone | 38.9 | 99 | 71 | 114 |
| 10 | δ-Undecanolactone | 41.7 | 85 | 55 | 128 |
| 11 | γ-Dodecalactone | 43.7 | 99 | 71 | 114 |
| 12 | δ-Dodecalactone | 46.0 | 85 | 69 | 128 |

In the present disclosure, calcium hydroxyphosphate microspheres are used as an adsorbent for the QuEChERS (quick, easy, cheap, effective, rugged, and safe) pretreatment of fruit juice. Acetonitrile is used as an organic solvent to extract flavor substances from fruit juice beverages, and then synthesized calcium hydroxyphosphate microspheres are used as an adsorption material to remove the matrixes (including interfering components such as pigments, fats, and proteins) from the extracted solution, obtaining purified test solution. The purified test solution is analyzed using GC-MS. By analyzing the spectra of chiral flavor substances and comparing the peak area ratios of different chiral configurations, it can be determined whether the chiral flavor substances are natural or artificial fragrances, thereby evaluating the quality of the fruit juice beverages.

The present disclosure uses a GC-MS method to identify whether artificial lactone compounds are added to a fruit juice. Chiral lactone fragrances are extracted into an organic solvent through a simple QuEChERS pretreatment step and are identified by comparing the ratios of different isomers of chiral lactones using chiral GC-MS, without requiring large amounts of organic solvents or derivatization, resulting in a simple operation process and greatly improved detection efficiency.

### BRIEF DESCRIPTION OF THE DRAWINGS

FIG. 1 shows an XRD (X-ray diffraction) spectrum of the hydroxyapatite prepared in Example 1;

FIG. 2A shows SEM (scanning electron microscope) images of the hydroxyapatite prepared in Example 1 (scale is 5 μm), FIG. 2B shows SEM images of the hydroxyapatite prepared in Example 1 (scale is 500 nm), FIG. 2C shows the scanning area for EDS (energy dispersive spectroscopy) of the hydroxyapatite prepared in Example 1, and FIG. 2D shows the elemental content of the area scanned in FIG. 2C for the hydroxyapatite prepared in Example 1;

FIG. 3 shows an FTIR (fourier transform infrared spectroscopy) spectrum of the hydroxyapatite prepared in Example 1;

FIG. 4 shows a Raman spectrum of the hydroxyapatite prepared in Example 1;

FIG. 5A shows a pore size distribution diagram of the hydroxyapatite prepared in Example 1, and FIG. 5B shows N₂ adsorption-desorption curves of the hydroxyapatite prepared in Example 1;

FIG. 6 shows pictures of the fruit juice to be tested in Example 1 and Comparative examples 1-3 before and after pretreatment, with the left panel showing a picture before the pretreatment, and the right panel showing a picture after the pretreatment, where test tube No. 1 corresponds to Comparative example 2, test tube No. 2 corresponds to Comparative Example 3, test tube No. 3 corresponds to Example 1, and test tube No. 4 corresponds to Comparative example 1;

FIG. 7 shows a total ion chromatogram obtained by GC-MS detection of the test solutions in Example 1 and Comparative examples 1-3;

FIG. 8 shows a total ion chromatogram obtained by detecting a mixed standard solution with a mass concentration of 100 mg/L under different split ratios;

FIG. 9 shows a total ion chromatogram obtained by detecting a mixed standard solution with a mass concentration of 100 mg/L under different temperature rise programs; and

FIG. 10 shows a total ion chromatogram obtained by detecting a mixed standard solution with a mass concentration of 100 mg/L using different types of chromatographic columns.

### DETAILED DESCRIPTION OF THE EMBODIMENTS

The present disclosure provides use of hydroxyapatite as an adsorbent in pretreatment of a sample when detecting the sample using GC-MS.

As a specific embodiment of the present disclosure, the hydroxyapatite is prepared by a process including:
mixing calcium chloride, diammonium hydrogen phosphate, a hydrochloric acid solution, citric acid, urea, and water, and conducting a hydrothermal reaction to obtain the hydroxyapatite.

As a specific embodiment of the present disclosure, the mixing includes the following steps:
dissolving the calcium chloride in the water to obtain a calcium chloride solution;
adding the diammonium hydrogen phosphate and the hydrochloric acid solution to the calcium chloride solution for first mixing to obtain a first mixed solution; and
adding citric acid monohydrate and the urea to the first mixed solution for second mixing.

As a specific embodiment of the present disclosure, the calcium chloride is calcium chloride dihydrate; and the calcium chloride solution has a molar concentration of 0.048 mol/L to 0.52 mol/L, and specifically 0.5 mol/L. There is no special requirement for the dissolving in the present disclosure, as long as the calcium chloride can be completely dissolved.

As a specific embodiment of the present disclosure, a molar ratio of the calcium chloride to the diammonium hydrogen phosphate is in a range of 8:4.7 to 8:4.9, and specifically is 8:4.8; the hydrochloric acid solution has a molar concentration of 0.8 mol/L to 1.2 mol/L, and specifically 1 mol/L; and pH of a resulting system after adding the hydrochloric acid solution is 2 to 3, or 2.5-3. There is no special requirement for the first mixing in the present disclosure, as long as uniform mixing can be achieved.

As a specific embodiment of the present disclosure, the citric acid is citric acid monohydrate, and a molar ratio of the calcium chloride to the citric acid monohydrate is in a range of 1.8:1 to 2.2:1, and specifically is 2:1; and a molar ratio of the calcium chloride to the urea is in a range of 1.8:1 to 2.2:1, and specifically is 2:1. As a specific embodiment of the present disclosure, the second mixing is conducted with stirring for 25 min to 35 min, or 28 min to 30 min; and there is no special requirement for the rotation speed of the stirring in the present disclosure, as long as uniform mixing can be achieved. In the present disclosure, the citric acid monohydrate is used as a growth directing agent, which enables the formation of spherical calcium hydroxyphosphate.

As a specific embodiment of the present disclosure, the hydrothermal reaction is conducted at a temperature of 100 °C to 140 °C, or 120 °C to 130 °C for 2 h to 4 h, or 3 h to 3.5 h.

As a specific embodiment of the present disclosure, after the hydrothermal reaction, the process for preparing the hydroxyapatite further includes: cooling a resulting system after the hydrothermal reaction to room temperature, and conducting solid-liquid separation, and washing and then drying a resulting solid after the solid-liquid separation to obtain the hydroxyapatite. As a specific embodiment of the present disclosure, the room temperature is 20 °C to 35 °C, or 25 °C to 30 °C; and the cooling process is not particularly limited, as long as it can reach the desired temperature. As a specific embodiment of the present disclosure, the solid-liquid separation is conducted by suction filtration; the washing is conducted by washing with water and ethanol in sequence; and the drying is conducted at a temperature of 55 °C to 60 °C; and there is no special requirement for the drying time in the present disclosure, as long as the solvent can be removed.

The present disclosure further provides a pretreatment method of a sample, including the following steps:
subjecting a fruit juice to be tested, acetonitrile, first magnesium sulfate, and sodium chloride to a first mixing, and then a first vortexing and a first centrifugation in sequence to obtain a first liquid phase;
subjecting the first liquid phase, second magnesium sulfate, and hydroxyapatite to a second mixing and then a second centrifugation to obtain a second liquid phase; and
filtering the second liquid phase through an organic filter membrane to obtain a test solution.

In the present disclosure, a fruit juice to be tested, acetonitrile, first magnesium sulfate, and sodium chloride are subjected to a first mixing, and then a first vortexing and a first centrifugation in sequence to obtain a first liquid phase. As a specific embodiment of the present disclosure, a ratio of a mass of the fruit juice to be tested to a volume of the acetonitrile is in a range of 1 g: 1.5 to 1 g: 2.5 mL, and specifically is 1 g: 1.5 mL, 1 g: 2 mL, or 1 g: 2.5 mL; a mass ratio of the fruit juice to be tested to the first magnesium sulfate is in a rang of 5:1.8 to 5:2.2, and specifically is 5:2; and a mass ratio of the fruit juice to be tested to the sodium chloride is in a range of 5:1.8 to 5:2.2, and specifically is 5:2. The first mixing is not particularly limited in the present disclosure, as long as uniform mixing can be achieved. As a specific embodiment of the present disclosure, the first vortexin is conducted for 0.8 min to 1.2 min, specifically 1 min; the first centrifugation is conducted at a rotation speed of 3,800 rpm to 4,200 rpm, or 3,900 rpm to 4,000 rpm; and the first centrifugation is conducted for 4 min to 6 min, or 4 min to 5 min. In the present disclosure, after the first centrifugation, a supernatant is obtained as the first liquid phase.

In the present disclosure, the first magnesium sulfate is used as a water removing agent, which can absorb moisture in the sample during the extraction process; and the sodium chloride facilitates the layering of the solution during the sample extraction process, promoting the separation of acetonitrile from the aqueous phase.

In the present disclosure, after obtaining the first liquid phase, the first liquid phase, second magnesium sulfate, and hydroxyapatite are subjected to a second mixing and then a second centrifugation to obtain a second liquid phase. As a specific embodiment of the present disclosure, a ratio of a mass of the second magnesium sulfate to a volume of the first liquid phase is in a range of 1 g: 8 mL to 1.4 g: 8 mL, or 1.2 g: 8 mL to 1.3 g: 8 mL; and a ratio of a mass of the hydroxyapatite to the volume of the first liquid phase is in a range of 380 mg: 8 mL to 420 mg: 8 mL, or 400-410 mg: 8 mL. In the present disclosure, the second magnesium sulfate is used as a water removing agent. As a specific embodiment of the present disclosure, the second mixing is conducted under conditions for the second vortexing, and the second vortexing is conducted for 25 s to 35 s, or 30 s to 33 s; and in the present disclosure, interfering components such as pigments, fats, and proteins in the first liquid phase are adsorbed by the hydroxyapatite during the second mixing process. As a specific embodiment of the present disclosure, the second centrifugation is conducted at a rotation speed of 3,800 rpm to 4,200 rpm, or 3,900 rpm to 4,000 rpm; and the second centrifugation is conducted for 4 min to 6 min, or 4 min to 5 min. In the present disclosure, after the second centrifugation, a supernatant is obtained as the second liquid phase.

In the present disclosure, after obtaining the second liquid phase, the second liquid phase is filtered through an organic filter membrane to obtain a test solution. As a specific embodiment of the present disclosure, the organic filter membrane has an average pore size of 0.2 μm to 0.24 μm, or 0.22 μm to 0.23 μm. In the present disclosure, insoluble impurities can be removed through filtration, thus preventing clogging of detection instruments (GC-MS being a precision instrument), reducing the occurrence of extraneous peaks that interfere with the sample, and ensuring the stability and repeatability of the instrument.

The present disclosure further provides a detection method of a lactone compound in a fruit juice, including the following steps:
subjecting a test solution to GC-MS detection to obtain a total ion chromatogram; and
obtaining a ratio of R configuration to S configuration of the lactone compound in the test solution based the total ion chromatogram; where
the test solution is the test solution obtained by the pretreatment method as described above;
the lactone compound includes at least one selected from the group consisting of γ-valerolactone, γ-caprolactone, γ-heptalactone, γ-octalactone, γ-nonalactone, δ-nonalactone, γ-decalactone, δ-decalactone, γ-undecanolactone, δ-undecanolactone, γ-dodecalactone, and δ-dodecalactone.

In the present disclosure, the test solution is subjected to GC-MS detection to obtain a total ion chromatogram. As a specific embodiment of the present disclosure, conditions for a gas chromatography detection in the GC-MS detection include: chromatographic column: CP-Chirasil Dex CB (length 25 m, internal diameter 0.25 mm, film thickness 0.25 μm); split ratio: 10:1; and temperature rise program: initial column temperature: 40 °C, increase to 80 °C at a heating rate of 10 °C/min, then to 180 °C at a heating rate of 2 °C/min, and then to 200 °C at a heating rate of 10 °C/min, and hold at the 200 °C for less than 20 min. The conditions for the gas chromatography detection further include: injection volume: 1 μL; constant flow mode, with helium as a carrier gas at a flow rate of 0.8 mL/min to 1.2 mL/min; inlet temperature: 220 °C to 250 °C; and transfer line temperature: 200 °C, where the flow rate is 1 mL/min. As a specific embodiment of the present disclosure, the CP-Chirasil Dex CB chromatographic column is purchased from Agilent Technologies, Co., Ltd. In some embodiments of the present disclosure, the CP-Chirasil Dex CB chromatographic column is capable of separating the R and S configurations of lactone compounds.

As a specific embodiment of the present disclosure, conditions for a mass chromatography detection in the GC-MS detection include: chromatographic column: CP-Chirasil Dex CB; using electron impact ionization, with an ionization energy of 70 eV; a temperature of an ion source and quadrupole rods being 230 °C and 150 °C, respectively; solvent delay: 5 min to 10 min; full scan mode with a mass scan range of 40 m/z to 400 m/z; and parameters for single ion monitoring mode being shown in Table 1:

TABLE 1
Parameters for single ion monitoring mode

| | Monitored | Start time | Quantitative | Qualitative | Qualitative |
|---|---|---|---|---|---|
| No. | compound | (min) | ion (m/z) | ion 1 (m/z) | ion 2 (m/z) |
| 1 | γ-Valerolactone | 10 | 56 | 41 | 85 |
| 2 | γ-Caprolactone | 15.3 | 85 | 57 | 70 |
| 3 | γ-Heptalactone | 20.8 | 85 | 56 | 100 |
| 4 | γ-Octalactone | 26 | 85 | 56 | 100 |
| 5 | γ-Nonalactone | 29.2 | 99 | 71 | 114 |
| 6 | δ-Nonalactone | 31.6 | 85 | 100 | 114 |
| 7 | γ-Decalactone | 34 | 99 | 71 | 114 |
| 8 | δ-Decalactone | 36.4 | 85 | 55 | 128 |
| 9 | γ-Undecanolactone | 38.9 | 99 | 71 | 114 |
| 10 | δ-Undecanolactone | 41.7 | 85 | 55 | 128 |
| 11 | γ-Dodecalactone | 43.7 | 99 | 71 | 114 |
| 12 | δ-Dodecalactone | 46 | 85 | 69 | 128 |

As a specific embodiment of the present disclosure, a working solution is prepared by a process including the following steps:
dissolving lactone compound standards in acetonitrile to obtain single standard solutions, respectively, where the lactone compound standards include γ-valerolactone, γ-caprolactone, γ-heptalactone, γ-octalactone, γ-nonalactone, δ-nonalactone, γ-decalactone, δ-decalactone, γ-undecanolactone, δ-undecanolactone, γ-dodecalactone, and δ-dodecalactone;
mixing twelve single standard solutions to obtain a mixed standard solution; and
diluting the mixed standard solution with acetonitrile to obtain the working solution.

As a specific embodiment of the present disclosure, the single standard solution has a mass concentration of 10,000 mg/L; the mixed standard solution has a mass concentration of 100 mg/L; and the working solution has a mass concentration of 0.1 mg/L, 0.2 mg/L, 0.5 mg/L, 1 mg/L, 2 mg/L, and 5 mg/L.

In the present disclosure, the conditions for GC-MS detection of the working solution may be consistent with the conditions used for the test solution, which would not be reiterated here to avoid redundancy.

In the present disclosure, after obtaining the total ion chromatogram, a ratio of R configuration to S configuration of the lactone compound in the test solution is obtained based on the total ion chromatogram. The present disclosure determines whether artificially synthesized fragrances are added to the test solution based on the R/S configuration ratio. Artificially synthesized lactone fragrances typically exhibit an R/S configuration ratio of lactone compounds close to 1:1. In contrast, in natural fruits, due to the stereoselectivity of enzymes, the synthesized lactone compounds often have one configuration that accounts for the majority or 100%.

To further illustrate the present disclosure, the technical solutions provided by the present disclosure will be described in detail below in conjunction with examples, which cannot be construed as limiting the scope of the present disclosure, however.

### Example 1

### Preparation of Hydroxyapatite:

0.008 mol of CaCl₂·2H₂O was dissolved in 160 mL of deionized water, and then 0.0048 mol of (NH₄)₂HPO₄ was added thereto and subjected to mixing until uniform; pH of a resulting system was adjusted to 2.5 using a hydrochloric acid solution with a molar concentration of 1 mol/L to enable complete dissolution, and then 0.004 mol of citric acid monohydrate was added thereto, followed by addition of 2.4 g of urea; a resulting solution was stirred for 30 min and then transferred into a 100 mL hydrothermal autoclave and subjected to a hydrothermal reaction at 120 °C for 3 h; and a resulting product was naturally cooled to 30 °C and was then subjected to suction filtration, and a solid obtained by the suction filtration was washed with water (2 L water) and ethanol in sequence, and then dried in an oven at 60 °C to obtain the hydroxyapatite, denoted as HAP-N.

FIG. 1 shows the XRD spectrum of the prepared hydroxyapatite. As can be seen from FIG. 1, the XRD spectrum of the prepared HAP-N is essentially consistent with that of the standard HAP (Ca₅(PO₄)₃OH, PDF #09-0432). No peaks corresponding to other calcium phosphate impurities are observed, indicating the successful preparation of pure-phase HAP. However, it is evident from the figure that the diffraction peaks of the synthesized HAP-N at 20 values of 31.761, 32.196, and 32.908 are relatively broad, suggesting that the crystallinity of HAP-N is relatively low. Poor crystallinity may contribute to an increased number of binding sites, thereby enhancing adsorption efficiency.

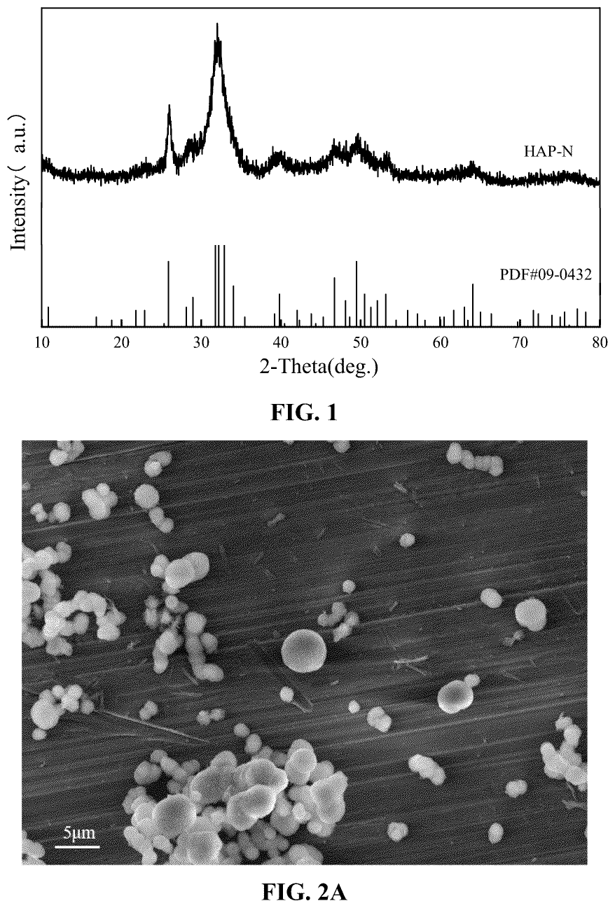

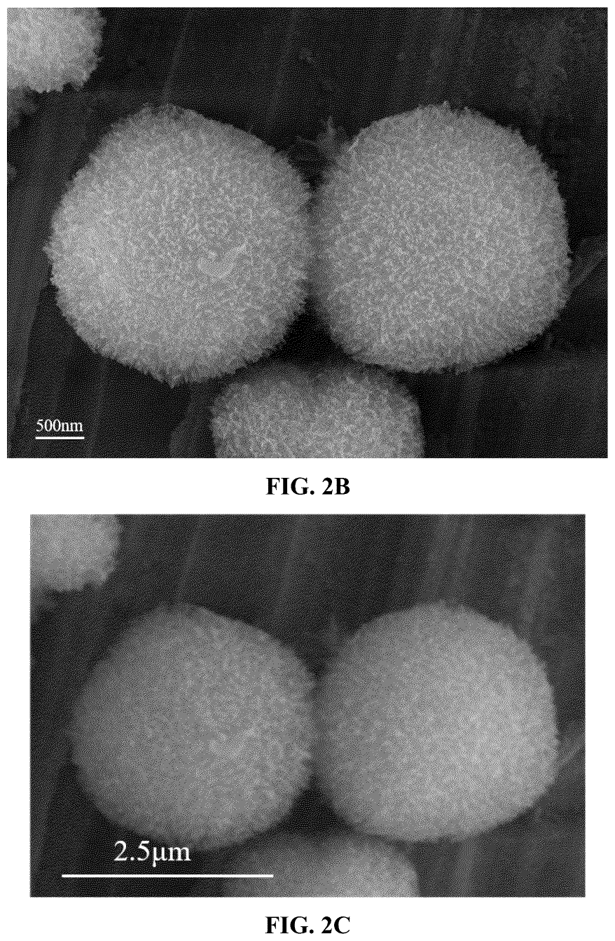

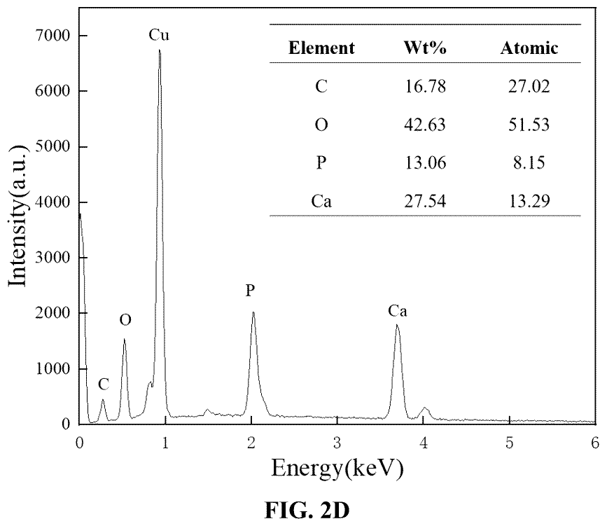

FIG. 2A to FIG. 2D show the SEM and EDS images of the prepared hydroxyapatite. Due to the addition of citric acid and urea, the SEM images of the sample were tested using a copper substrate. FIG. 2A to FIG. 2B are SEM images at different magnifications, and FIG. 2C to FIG. 2D are EDS images. From FIG. 2A to FIG. 2B, it can be observed that the prepared HAP-N exhibits a spherical morphology with slight surface protrusions, and some particles appear aggregated. The prepared hydroxyapatite has a diameter in a range of 2 μm to 5 μm and demonstrates good dispersibility. This spherical particle structure, combined with high dispersibility, leads to an increased specific surface area of the prepared hydroxyapatite. As can be seen from FIG. 2D, the synthesized HAP-N mainly contains C, O, P, and Ca (H generally cannot be detected by EDS). Combined with XRD results, this further confirms that the synthesized product is pure-phase HAP. Furthermore, the sample contains a significant amount of carbon, likely due to the addition of urea and citric acid. However, according to the XRD results, this portion of carbon does not cause any phase change in hydroxyapatite.

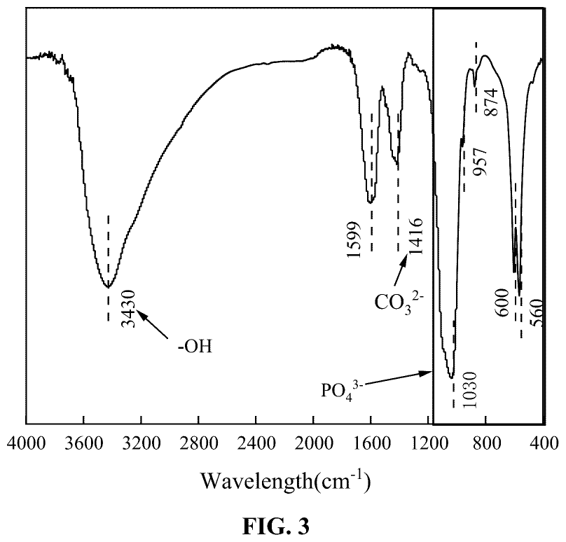

FIG. 3 shows the FTIR spectrum of the prepared hydroxyapatite HAP-N. As can be seen from FIG. 3, the absorption peak at 3430 cm⁻¹ is attributed to the stretching vibration of the OH⁻ group in hydroxyapatite, and the absorption peak at 1599 cm⁻¹ is attributed to the stretching vibration of the C═O group in the urea molecule. The absorption peaks at 1030 cm⁻¹, 957 cm⁻¹, 600 cm⁻¹, and 560 cm⁻¹ are caused by the vibration of the PO₄³⁻ group. The infrared data further indicates that citric acid is not present in the final synthesized phase, suggesting that during the synthesis process, citric acid, on the one hand, facilitates the oriented crystal growth, and on the other hand, enhances the dispersibility of HAP-N. These effects collectively increase the specific surface area, which is highly beneficial for adsorption performance.

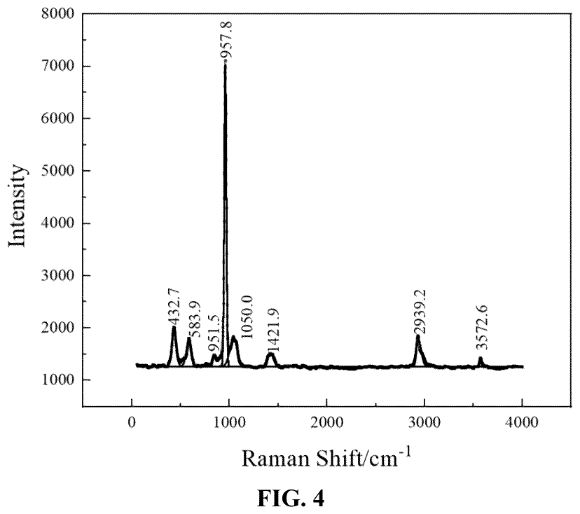

Raman spectroscopy, compared to Fourier-transform infrared spectroscopy, offers higher resolution and can provide further clarification on the composition of functional groups. FIG. 4 shows the Raman spectrum of the prepared hydroxyapatite HAP-N. As can be seen from FIG. 4, the v1 band of PO₄³⁻ at 957.8 cm⁻¹ exhibits the highest intensity, and the v2 band of PO₄³⁻ group is observed at 432.7 cm⁻¹, the v3 band of PO₄³⁻ group is observed at 1050 cm⁻¹, and the v4 band of PO₄³⁻ is observed at 583.9 cm⁻¹. The stretching vibration band of the hydroxyl group is observed at 3572.6 cm⁻¹. Additionally, for the sample, new peaks appear at 951.5 cm⁻¹ and 2939.2 cm⁻¹. Based on the analysis of relevant literature and XRD results, the peak at 2939.2 cm⁻¹ is likely due to a small amount of calcium-deficient hydroxyapatite in HAP-N.

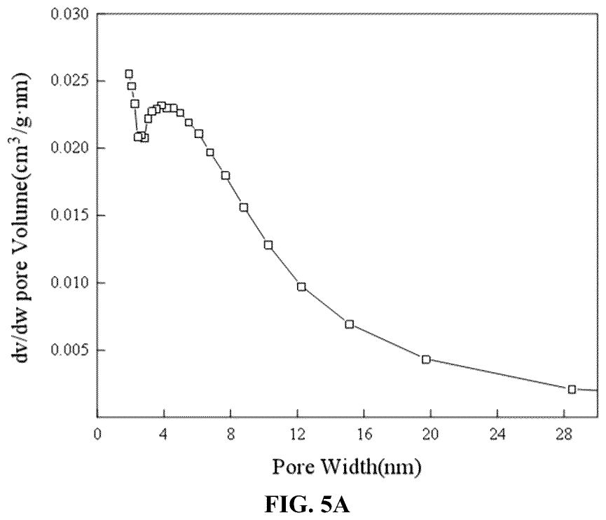

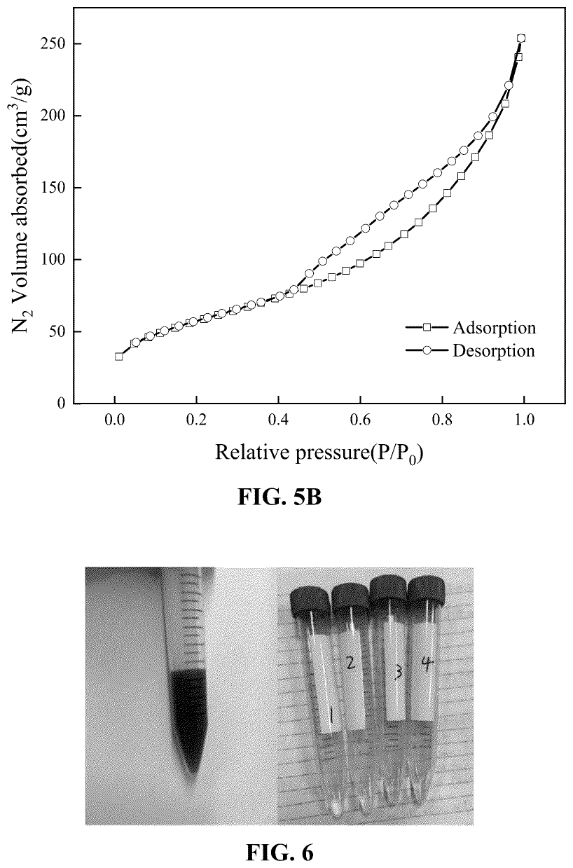

In SEM, the inventor has observed that hydroxyapatite exhibits a spherical morphology with good dispersibility. Therefore, to determine whether this morphology leads to an increased specific surface area, the BET of HAP-N was tested. FIGS. 5A-5B show the N₂ adsorption-desorption curves and a pore size distribution diagram of the prepared hydroxyapatite HAP-N. The BET test indicates that HAP-N exhibits a type IV isotherm, which is characteristic of mesoporous materials, and exhibits an H₃-type hysteresis loop. The specific surface area of HAP-N, calculated using BET is 206.8657 m²/g, which is higher than that of other HAP materials. Pore size distribution indicates that the pore size of HAP-N is predominantly in a range of 3 nm to 8 nm, belonging to mesoporous materials, which is consistent with the results shown by the type IV isotherm observed.

### Pretreatment of Fruit Juice:

A fruit juice to be tested was Naturalis strawberry compound juice (strawberry compound juice containing no added essence) purchased from a supermarket.

5.0 g of the fruit juice to be tested was mixed with 10 ml of acetonitrile and stirred for 30 s until uniform, then 2 g of MgSO₄ and 2 g of NaCl were added thereto and subjected to vortex extraction for 1 min, followed by centrifugation at 4000 rpm for 5 min, and a supernatant was obtained as a first liquid phase. 8 mL of the first liquid phase, 1.2 g of MgSO₄, and 400 mg of HAP-N were mixed in a vortex manner for 30 s, followed by centrifugation at 4000 rpm for 5 min, and a supernatant was obtained as a second liquid phase. The second liquid phase was filtered through an organic phase filter membrane with a pore size of 0.22 μm to obtain a test solution. GC-MS detection of test solution:

Twelve lactone compound standards, consisting of a γ-valerolactone standard, a γ-caprolactone standard, a γ-heptalactone standard, a γ-octalactone standard, a γ-nonalactone standard, a δ-nonalactone standard, a γ-decalactone standard, a δ-decalactone standard, a γ-undecanolactone standard, a δ-undecanolactone standard, a γ-dodecalactone standard, and a δ-dodecalactone standard, were dissolved in acetonitrile to obtain single standard solutions with a mass concentration of 10,000 mg/L. Twelve single standard solutions were mixed and then diluted with acetonitrile to obtain a mixed standard solution with a mass concentration of 100 mg/L. The mixed standard solution with the mass concentration of 100 mg/L was tested in full scan mode to obtain the retention time and mass spectrometry information of the twelve lactones, which were used as parameters for setting up single ion monitoring (SIM) mode. The results are listed in Table 1.

The test solution was subjected to GC-MS detection to obtain a total ion chromatogram. A ratio of R configuration to S configuration of the lactone compound in the test solution was obtained based on the total ion chromatogram; where conditions for a gas chromatography detection in the GC-MS detection were as follows: chromatographic column: CP-Chirasil Dex CB purchased from Agilent Technologies, Co., Ltd.; split ratio: 10:1; and temperature rise program: initial column temperature: 40 °C, increase to 80 °C at a heating rate of 10 °C/min, then to 180 °C at a heating rate of 2 °C/min, and then to 200 °C at a heating rate of 10 °C/min, and hold at the 200 °C for 20 min; injection volume: 1 μL; constant flow mode, with helium as a carrier gas at a flow rate of 1.0 mL/min; inlet temperature: 250 °C; and transfer line temperature: 200 °C. Conditions for a mass spectrometry detection in the GC-MS detection were as follows: chromatographic column: CP-Chirasil Dex CB purchased from Agilent Technologies, Co., Ltd.; using electron impact ionization, with an ionization energy of 70 eV; a temperature of an ion source and quadrupole rods being 230 °C and 150 °C, respectively; solvent delay: 10 min; full scan mode with a mass scan range of 40 m/z to 400 m/z; and parameters for single ion monitoring mode being shown in Table 1.

Method for detecting the content of lactone compounds in a test solution: a mixed standard solution with a mass concentration of 100 mg/L was diluted using acetonitrile to prepare a series of working solutions with mass concentrations of 0.1 mg/L, 0.2 mg/L, 0.5 mg/L, 0.7 mg/L, and 1 mg/L, which were subjected to GC-MS detection according to the method described above to obtain a total ion chromatogram. A standard curve was established based on the peak areas (integrated areas) in the total ion chromatogram and the corresponding mass concentration values, as detailed in Table 2, where x represents the concentration of a lactone compound, and y represents the peak area of the corresponding lactone compound. The peak areas of the lactone compounds can be determined based on the total ion chromatogram obtained from the detection of the test solution, and by substituting these peak areas into the standard curve, the concentrations of the corresponding lactone compounds in the test solution can be calculated.

TABLE 2
Regression equations, linear ranges, and correlation
coefficients of twelve lactone compounds

| | | Linear range | | Correlation |
|---|---|---|---|---|
| No | Compound | (μg/L) | Regression equation | coefficient |
| 1 | γ-Valerolactone | 0.1-1 | y = 13351.773325*x + | 0.999 |
| | | | 146.104838 | |
| 2 | γ-Caprolactone | 0.1-1 | y = 22000.117289*x + | 0.999 |
| | | | 5050.643184 | |
| 3 | γ-Heptalactone | 0.1-1 | y = 24156.796989*x + | 0.999 |
| | | | 6014.280298 | |
| 4 | γ-Octalactone | 0.1-1 | y = 22305.805028*x + | 0.999 |
| | | | 4646.125522 | |
| 5 | γ-Nonalactone | 0.1-1 | y = 18520.156400*x + | 0.999 |
| | | | 4698.632951 | |
| 6 | δ-Nonalactone | 0.1-1 | y = 6586.864945*x + | 0.999 |
| | | | 3519.846459 | |
| 7 | γ-Decalactone | 0.1-1 | y = 14646.885921*x + | 0.999 |
| | | | 5786.898477 | |
| 8 | γ-Decalactone | 0.1-1 | y = 6326.572917*x + | 0.999 |
| | | | 4002.901554 | |
| 9 | γ-Undecanolactone | 0.1-1 | y = 10950.552010*x + | 0.999 |
| | | | 5395.248436 | |
| 10 | δ-Undecanolactone | 0.1-1 | y = 5153.737937*x + | 0.999 |
| | | | 3597.664961 | |
| 11 | γ-Dodecalactone | 0.1-1 | y = 7741.354237*x + | 0.999 |
| | | | 4616.143648 | |
| 12 | δ-Dodecalactone | 0.1-1 | y = 4248.456560*x + | 0.999 |
| | | | 2931.817065 | |

### Comparative Example 1

Hydroxyapatite was prepared according to the method in Example 1, except that 0.004 mol of citric acid monohydrate was replaced with 0.00015 mol of ethylenediaminetetraacetic acid (EDTA), and a resulting hydroxyapatite was denoted as HA-EDTA.

A fruit juice to be tested was pretreated according to the method in Example 1 to obtain a test solution, except that 400 mg of HAP-N was replaced with 400 mg of HA-EDTA.

The test solution was subjected to GC-MS detection according to the method in Example 1 to obtain a total ion chromatogram.

### Comparative Example 2

A fruit juice to be tested was pretreated according to the method in Example 1 to obtain a test solution, except that 400 mg of HAP-N was replaced with a commercially available salt packet (which was denoted as salt packet No. 1), where the salt packet No. 1 consisted of 1.2 mg of MgSO₄ and 400 mg of N-propylethylenediamine (PSA).

The test solution was subjected to GC-MS detection according to the method in Example 1 to obtain a total ion chromatogram.

### Comparative Example 3

A fruit juice to be tested was pretreated according to the method in Example 1 to obtain a test solution, except that 400 mg of HAP-N was replaced with a commercially available salt packet (which was denoted as salt packet No. 2), where the salt packet No. 2 consisted of 1.2 mg of MgSO₄, 400 mg of N-propylethylenediamine (PSA), 200 mg of graphitized carbon black (GCB), and 400 mg of octadecylsilane (C18).

The test solution was subjected to GC-MS detection according to the method in Example 1 to obtain a total ion chromatogram.

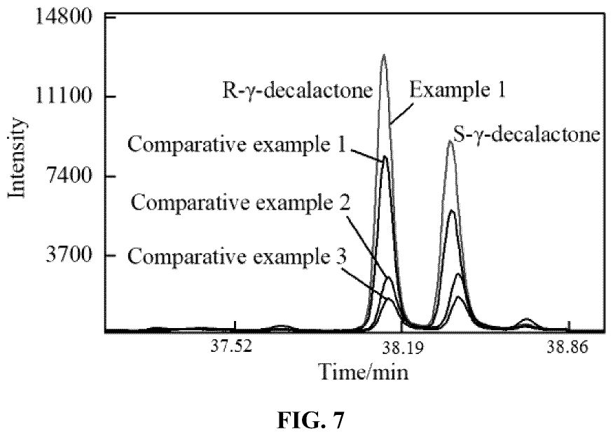

FIG. 6 shows pictures of the fruit juice to be tested in Example 1 and Comparative examples 1-3 before and after pretreatment, with the left panel showing a picture before the pretreatment, and the right panel showing a picture after the pretreatment, where test tube No. 1 corresponds to Comparative example 2, test tube No. 2 corresponds to Comparative Example 3, test tube No. 3 corresponds to Example 1, and test tube No. 4 corresponds to Comparative example 1. FIG. 7 shows a total ion chromatogram obtained by GC-MS detection of the test solutions in Example 1 and Comparative examples 1-3.

As observed in FIG. 7, γ-decalactone exhibits chromatographic peaks for both the R and S isomers within the monitoring range of 36.4 min to 38.9 min, indicating that this fruit juice product, which is labeled as containing no added essence, actually uses a chemically synthesized γ-decalactone as a fragrance (since natural strawberries only contain the R configuration of γ-decalactone). In addition, the abundance of γ-decalactone in Example 1 and Comparative example 1 is higher than that in Comparative examples 2 and Comparative examples 3, indicating that the HAP-N and HA-EDTA materials have a lower adsorption capacity for the target lactone compounds compared to the adsorbents in the salt package No. 1 and salt package No. 2, resulting in more lactone compounds in the extracted solution. Thus, using the HAP-N material as an adsorbent can effectively adsorb impurities and help identify the adulteration of natural fruit juice with synthetic fragrances, and the synthesis cost is lower.

The concentration of γ-decalactone in the fruit juice to be tested of Example 1 and Comparative examples 1-3 was determined using the method described above for measuring the content of lactone compounds in test solutions. The peak areas obtained from the total ion chromatograms were substituted into the corresponding standard curves to calculate the concentration of γ-decalactone. The results for Example 1 and Comparative examples 1-3 are 6.32 μg/L, 3.84 μg/L, 1.56 μg/L, and 0.59 μg/L, respectively.

### Test Example 1

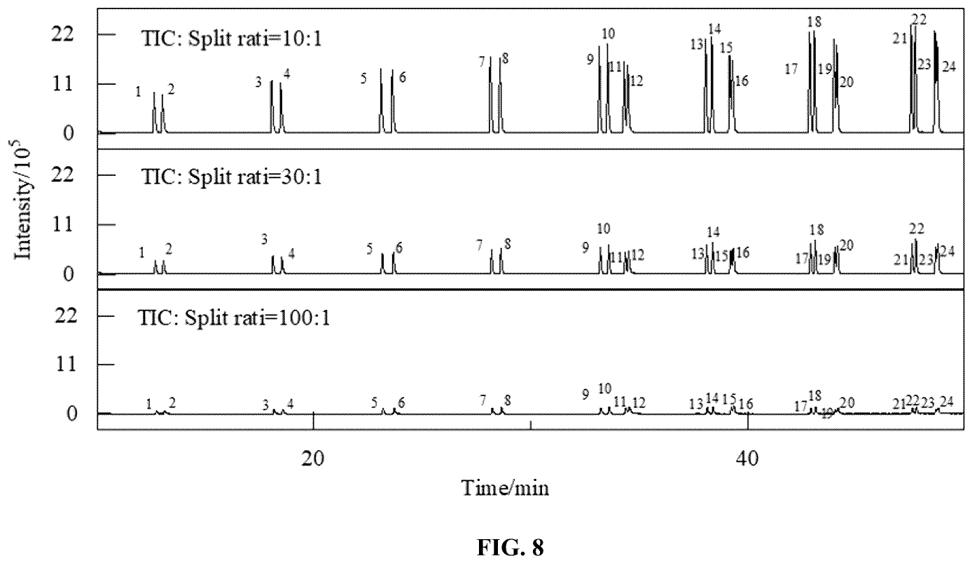

The mixed standard solution with the mass concentration of 100 mg/L in Example 1 was subjected to GC-MS detection as described in Example 1. The GC-MS detection conditions were the same as those in Example 1, except for the split ratio. In full scan mode, the mixed standard solution with the mass concentration of 100 mg/L was tested in three different injection modes with split ratios of 10:1 (v:v), 30:1 (v:v), and 100:1 (v:v), respectively. The total ion chromatograms obtained from these tests are shown in FIG. 8, where 1 is R-γ-valerolactone, 2 is S-γ-valerolactone, 3 is R-γ-caprolactone, 4 is S-γ-caprolactone, 5 is R-γ-heptalactone, 6 is S-γ-heptalactone, 7 is R-γ-octalactone, 8 is S-γ-octalactone, 9 is R-γ-nonalactone, 10 is S-γ-nonalactone, 11 is R-δ-nonalactone, 12 is S-δ-nonalactone, 13 is R-γ-decalactone, 14 is S-γ-decalactone, 15 is R-δ-decalactone, 16 is S-δ-decalactone, 17 is R-γ-undecanolactone, 18 is S-γ-undecanolactone, 19 is R-δ-undecanolactone, 20 is S-δ-undecanolactone, 21 is R-γ-dodecalactone, 22 is S-γ-dodecalactone, 23 is R-δ-dodecalactone, and 24 is S-δ-dodecalactone. The results reveal that at a split ratio of 10:1 (v:v), the responses of the twelve lactone compounds are the highest, with good resolution and well-defined peak shapes.

### Test Example 2

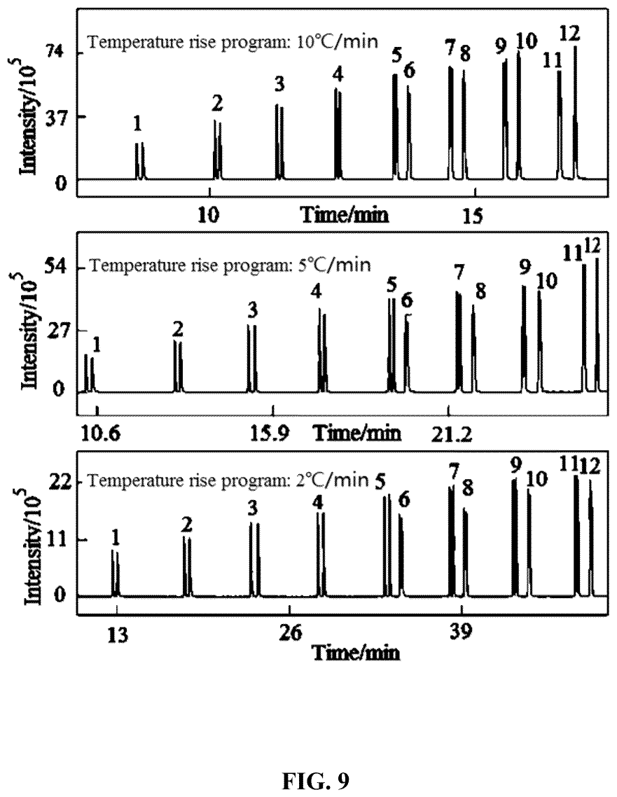

The mixed standard solution with the mass concentration of 100 mg/L in Example 1 was subjected to GC-MS detection as described in Example 1. The GC-MS detection conditions were the same as those in Example 1, except for the temperature rise program. The temperature was increased at a heating rate of 10 °C/min, 5 °C/min, and 2 °C/min in separate experiments within the range of 80 °C to 180 °C. The total ion chromatogram obtained under the condition of a heating rate of 10 °C/min is shown in FIG. 9, where 1 is γ-valerolactone, 2 is γ-caprolactone, 3 is γ-heptalactone, 4 is γ-octalactone, 5 is γ-nonalactone, 6 is 8-nonalactone, 7 is γ-decalactone, 8 is 8-decalactone, 9 is γ-undecanolactone, 10 is 8-undecanolactone, 11 is γ-dodecalactone, and 12 is the separated R and S configurations, with the left side showing the R configuration, and the right side showing the S configuration. Experimental results indicate that within this temperature range, when heated at a rate of 10 °C/min, the resolution of the two isomers for the compounds including 8-nonalactone, δ-decalactone, δ-undecalactone, γ-dodecalactone, and δ-dodecalactone is poor; when heated at a rate of 5 °C/min, the resolution of the two isomers for the compounds including 8-decalactone, δ-undecalactone, and δ-dodecalactone is poor; and when heated at a rate of 2 °C/min, all compounds exhibit good resolution.

### Test Example 3

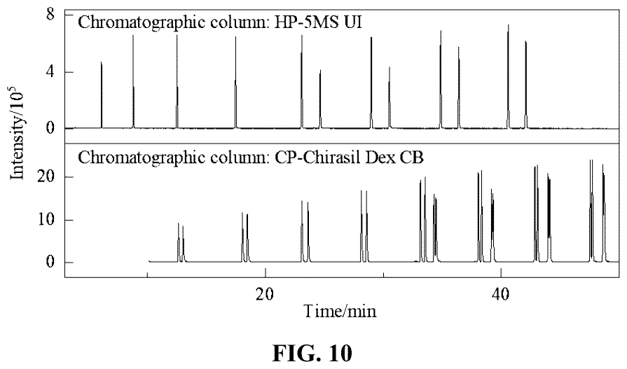

The mixed standard solution with the mass concentration of 100 mg/L in Example 1 was subjected to GC-MS detection as described in Example 1. The GC-MS detection conditions were the same as those in Example 1, except for the type of chromatographic columns. The total ion chromatograms obtained after the detection using a non-polar column HP-5 MS UI and a chiral column CP-Chirasil Dex CB, respectively, are shown in FIG. 10. Results indicate that the HP-5 MS UI chromatographic column fails to achieve chiral resolution of chiral lactone compounds.

In the present disclosure, the method for identifying whether an NFC fruit juice is adulterated with artificial lactone essence includes two steps: pretreatment and detection. The pretreatment step requires only simple equipment and operational steps, offering advantages such as low solvent consumption, minimal environmental pollution, and rapid analysis. For the detection, the method employs a chiral GC-MS system capable of separating isomers of chiral lactone compounds, eliminating the need for chemical/physical separation methods and thereby accelerating the experimental analysis process.

Although the embodiments described above provide a detailed description of the present disclosure, they are only some, rather than all of the embodiments of the present disclosure. All other embodiments that can be obtained according to the embodiments of the present disclosure without involving any inventive effort shall fall within the scope of the present disclosure.

What is claimed is:

1. A pretreatment method of a sample, comprising:
subjecting a fruit juice to be tested, acetonitrile, first magnesium sulfate, and sodium chloride to a first mixing, and then a first vortexing and a first centrifugation in sequence to obtain a first liquid phase;
subjecting the first liquid phase, second magnesium sulfate, and hydroxyapatite to a second mixing, and then a second vortexing and a second centrifugation in sequence to obtain a second liquid phase; and
filtering the second liquid phase through an organic filter membrane to obtain a test solution.

2. The pretreatment method of claim 1, wherein a mass ratio of the fruit juice to be tested to the first magnesium sulfate is in a range of 5:1.8 to 5:2.2; and
a mass ratio of the fruit juice to be tested to the sodium chloride is in a range of 5:1.8 to 5:2.2.

3. The pretreatment method of claim 1, wherein the first vortexing is conducted for 0.8 minutes to 1.2 minutes; and
the first centrifugation is conducted at a rotation speed of 3,800 rpm to 4,200 rpm for 4 minutes to 6 minutes.

4. The pretreatment method of claim 1, wherein a ratio of a mass of the second magnesium sulfate to a volume of the first liquid phase is in a range of 1 g: 8 mL to 1.4 g: 8 mL; and
a ratio of a mass of the hydroxyapatite to the volume of the first liquid phase is in a range of 380 mg: 8 mL to 420 mg: 8 mL.

5. The pretreatment method of claim 1, wherein the second vortexing is conducted for 25 seconds to 35 seconds; and
the second centrifugation is conducted at a rotation speed of 3,800 rpm to 4,200 rpm for 4 minutes to 6 minutes.

6. The pretreatment method of claim 1, wherein the organic filter membrane has an average pore size of 0.2 μm to 0.24 μm.

7. A detection method of a lactone compound in a fruit juice, comprising:
subjecting a test solution to gas chromatography-mass spectrometry detection to obtain a total ion chromatogram; and
obtaining a ratio of R configuration to S configuration of the lactone compound in the test solution based on the total ion chromatogram; wherein
the test solution is the test solution obtained by the pretreatment method of claim 1;
the lactone compound comprises at least one selected from the group consisting of γ-valerolactone, γ-caprolactone, γ-heptalactone, γ-octalactone, γ-nonalactone, δ-nonalactone, γ-decalactone, δ-decalactone, γ-undecanolactone, δ-undecanolactone, γ-dodecalactone, and δ-dodecalactone; and
conditions for a gas chromatography detection in the gas chromatography-mass spectrometry detection comprise: chromatographic column: CP-Chirasil Dex CB; split ratio: 10:1; and temperature rise program: initial column temperature: 40 °C, increase to 80 °C at a heating rate of 10 °C/min, then to 180 °C at a heating rate of 2 °C/min, and then to 200 °C at a heating rate of 10 °C/min, and hold at the 200 °C for less than 20 minutes.

8. The detection method of claim 7, wherein the conditions for the gas chromatography detection further comprise: injection volume: 1 μL; constant flow mode, with helium as a carrier gas at a flow rate of 0.8 mL/min to 1.2 mL/min; inlet temperature: 220 °C to 250 °C; and transfer line temperature: 200 °C.

9. The detection method of claim 7, wherein conditions for a mass spectrometry detection in the gas chromatography-mass spectrometry detection comprise: using electron impact ionization, with an ionization energy of 70 eV; a temperature of an ion source and quadrupole rods being 230 °C and 150 °C, respectively; solvent delay: 5 minutes to 10 minutes; full scan mode with a mass scan range of 40 m/z to 400 m/z; and parameters for single ion monitoring mode being shown in Table 1:
TABLE 1
Parameters for single ion monitoring mode

| | Monitored | Start time | Quantitative | Qualitative | Qualitative |
|---|---|---|---|---|---|
| No. | compound | (min) | ion (m/z) | ion 1 (m/z) | ion 2 (m/z) |
| 1 | γ-Valerolactone | 10.0 | 56 | 41 | 85 |
| 2 | γ-Caprolactone | 15.3 | 85 | 57 | 70 |
| 3 | γ-Heptalactone | 20.8 | 85 | 56 | 100 |
| 4 | γ-Octalactone | 26.0 | 85 | 56 | 100 |
| 5 | γ-Nonalactone | 29.2 | 99 | 71 | 114 |
| 6 | δ-Nonalactone | 31.6 | 85 | 100 | 114 |
| 7 | γ-Decalactone | 34.0 | 99 | 71 | 114 |
| 8 | δ-Decalactone | 36.4 | 85 | 55 | 128 |
| 9 | γ-Undecanolactone | 38.9 | 99 | 71 | 114 |
| 10 | δ-Undecanolactone | 41.7 | 85 | 55 | 128 |
| 11 | γ-Dodecalactone | 43.7 | 99 | 71 | 114 |
| 12 | δ-Dodecalactone | 46.0 | 85 | 69 | 128 |

10. The pretreatment method of claim 2, wherein the first vortexing is conducted for 0.8 minutes to 1.2 minutes; and
the first centrifugation is conducted at a rotation speed of 3,800 rpm to 4,200 rpm for 4 minutes to 6 minutes.

11. The pretreatment method of claim 4, wherein the second vortexing is conducted for 25 seconds to 35 seconds; and
the second centrifugation is conducted at a rotation speed of 3,800 rpm to 4,200 rpm for 4 minutes to 6 minutes.

12. The detection method of claim 7, a mass ratio of the fruit juice to be tested to the first magnesium sulfate is in a range of 5:1.8 to 5:2.2; and
a mass ratio of the fruit juice to be tested to the sodium chloride is in a range of 5:1.8 to 5:2.2.

13. The detection method of claim 12, wherein the first vortexing is conducted for 0.8 minutes to 1.2 minutes; and
the first centrifugation is conducted at a rotation speed of 3,800 rpm to 4,200 rpm for 4 minutes to 6 minutes.

14. The detection method of claim 7, wherein a ratio of a mass of the second magnesium sulfate to a volume of the first liquid phase is in a range of 1 g: 8 mL to 1.4 g: 8 mL; and
a ratio of a mass of the hydroxyapatite to the volume of the first liquid phase is in a range of 380 mg: 8 mL to 420 mg: 8 mL.

15. The detection method of claim 7, the second vortexing is conducted for 25 seconds to 35 seconds; and
the second centrifugation is conducted at a rotation speed of 3,800 rpm to 4,200 rpm for 4 minutes to 6 minutes.

16. The detection method of claim 7, wherein the organic filter membrane has an average pore size of 0.2 μm to 0.24 μm.
